(19) United States
(12) Patent Application Publication
(10) Pub. No.: US 2020/0038861 A1
(43) Pub. Date: Feb. 6, 2020

(54) SYSTEMS AND METHODS FOR HIGH THROUGHPUT SCREENING

(71) Applicant: THE REGENTS OF THE UNIVERSITY OF MICHIGAN, Ann Arbor, MI (US)

(72) Inventors: Euisik YOON, Superior Township, MI (US); Zhixiong ZHANG, Ann Arbor, MI (US); Yu-Chih CHEN, Ann Arbor, MI (US)

(21) Appl. No.: 16/339,872

(22) PCT Filed: Oct. 5, 2017
(86) PCT No.: PCT/US2017/055315

§ 371 Date: Apr. 5, 2019

Related U.S. Application Data
(60) Provisional application No. 62/405,671, filed on Oct. 7, 2016.

(51) Int. Cl. B01L 3/00; C12M 3/06; C12M 1/00; C12M 1/06; G01N 33/50; B01F 13/00; B01F 15/04

(52) U.S. Cl. CPC B01L 3/502715; C12M 23/16; C12M 23/20; C12M 27/02; G01N 33/5011; B01F 13/0061; B01F 2215/0073; B01L 2200/0694; B01L 2300/0867; B01L 2300/0809; B01L 2300/16; B01L 2300/0848; B01F 2215/0037; B01F 15/0404

(57) ABSTRACT

Provided herein are compositions, systems, and methods for high throughput screening. In particular, provided herein are microfluidic devices for high throughput analysis of multiplex chemical (e.g., drug interactions) across a wide range of concentrations.

### CROSS-REFERENCE TO RELATED APPLICATIONS

This application claims the benefit of U.S. provisional application Ser. No. 62/405,671, filed Oct. 7, 2016, which is incorporated herein by reference in its entirety.

### STATEMENT REGARDING FEDERALLY SPONSORED RESEARCH OR DEVELOPMENT

This invention was made with government support under W911NF-08-2-0004 awarded by the U.S. Army Research Laboratory, Army Research Office. The government has certain rights in the invention.

### FIELD OF THE INVENTION

Provided herein are compositions, systems, and methods for high throughput screening. In particular, provided herein are microfluidic devices for high throughput analysis of multiplex chemical (e.g., drug interactions) across a wide range of concentrations.

### BACKGROUND OF THE INVENTION

Cancer is the second most common cause of death in the United States, exceeded only by heart disease. In the United States, cancer accounts for 1 of every 4 deaths. The 5-year relative survival rate for all cancers patients diagnosed in 1996-2003 is 66%, up from 50% in 1975-1977 (see, e.g., Cancer Facts &Figures American Cancer Society: Atlanta, Ga. (2008)). This improvement in survival reflects progress in diagnosing at an earlier stage and improvements in treatment.

Considerable efforts have been made in precision cancer therapy in recent decades, which aims to customize appropriate treatment and decisions based on individual responses (Gonzalez de Castro, D., et al. Clinical Pharmacology & Therapeutics 93.3 (2013): 252-259). Although an increasing number of new cancer drugs have been developed as inhibitors of oncogenic drivers, clinical failures are still common due to drug resistance (Burrell, Rebecca A., et al., Molecular oncology 8.6 (2014): 1095-1111), which could be caused by the activity of alternative signaling pathways that compensate for the pathways being attacked (Zimmermann, Grant R., et al., Drug discovery today 12.1 (2007): 34-42). In order to overcome the limitations of mono-drug therapies, drug combinations have been widely accepted in cancer treatment for better therapeutic efficacy, which aims to inhibit multiple parallel redundant pathways for tumor cells to escape drug treatment (Devita, Vincent T., et al., Cancer 35.1 (1975): 98-110). Though drug combinations have high potential in cancer treatment, it remains challenging to efficiently identify effective combinations with a small number of patient primary samples for testing (Arnedos, Monica, et al. Molecular oncology 6.2 (2012): 204-210).

Due to the high cost of phase II and phase III clinical trials in drug development, in-vitro drug screening has been the gold standard in industry (Visk, DeeAnn. Applied In Vitro Toxicology 1.1 (2015): 79-82). High throughput screening accounts for a large share of the global drug discovery technologies market with an expected share of about 30% in 2015 valued at $18 billion followed by bioanalytical assays or instruments by about $14 billion. To pinpoint potent drug combinations, pharmaceutical companies are screening a large number of drug candidates, yet the resulting experimental complexity and cost increases exponentially (Macarron, Ricardo, et al. Nature reviews Drug discovery 10.3 (2011): 188-195). For example, investigation of 50 different compounds in pairwise combination yields ₅₀C₂=1225 different combinations. Furthermore, 7 different concentration ratios are used for each combination, and 5 replicates are typically used for each treatment condition. Considering a typical screening usually requires 15 different cell lines, which requires 1225×7×5×15 =643,125 wells for a typical test panel of well-plates, which is not only costly but also time consuming. The US Food and Drug Administration have expressed their dedication in developing novel combinatorial therapies, highlighting the need for innovative technologies to accelerate the discovery of novel drug combinations (US Food and Drug Administration (2010) “Guidance for Industry: Codevelopment of Two or More New Investigational Drugs for Use in Combination.”). To achieve high-throughput drug combination screening, several systems have been presented incorporating robotics and automatic handling. However, they are limited by complicated operation systems (Du, Guan-Sheng, et al. Analytical chemistry, 85(14), 6740-6747 (2013)) and time-consuming serial processes (Griner, Lesley A. Mathews, et al. Proc. Natl. Acad. Sci., 111(6), 2349-2354 (2014)).

Microfluidics emerges as a promising technology for both clinical precision medicine and industrial-scale drug discovery, thanks to its capability of handling small samples and highly multiplexed operations for high-throughput assays. Previous microfluidic high-throughput drug screening platforms introduce a “branching tree structure” to generate a linear drug concentration gradient, but they are limited to a combination of two drugs (Kim, Jeongyun, et al. Lab Chip 12.10 (2012): 1813-1822; An, D., et al., J. Biomolecules & therapeutics, 22(4), 355 (2014)). In addition, since cells respond to different drug concentrations in a non-linear manner, most drug screening methods require testing dosages ranging several orders of magnitude to calculate the 50% inhibition concentration (IC50) (Yun, Jae Young, et al. Analytical chemistry 83.16 (2011): 6148-6153). Conventional microfluidic gradient generators provide a linear concentration gradient using a “Christmas tree structure” (Chung, Bong Geun, et al., Lab on a Chip 5.4 (2005): 401-406). The narrow concentration range of conventional linear gradient generators severely hinders the use of microfluidics in drug screening.

Improved methods to identify cancer treatments, in particular combination treatments, are needed.

### SUMMARY OF THE INVENTION

Provided herein are compositions, systems, and methods for high throughput screening. In particular, provided herein are microfluidic devices for high throughput analysis of multiplex chemical (e.g., drug interactions) across a wide range of concentrations.

For example, in some embodiments, provided herein is a microfluidic device, comprising: i) a routing layer comprising a plurality of microfluidic channels and a plurality of chemical (e.g., drug) reservoirs, wherein the microfluidic channels are in fluid communication with the chemical reservoirs; ii) a mixing layer comprising a plurality of biased tree mixers patterned therein and a plurality of sphere culture chambers; and iii) a lid layer comprising a plurality of chemical inlet holes, a plurality of cell inlet holes, and a plurality of vias. In some embodiments, the vias are microfluidic channels that connect the lid, the mixing layer, and the routing layer. In some embodiments, the routing layer forms the bottom layer of the device, the mixing layer forms the middle layer of the device, and the lid layer forms the top layer of the device. In some embodiments, the microfluidic channels are 100 to 1000 μm in width and 100 to 1000 μm in height (e.g., approximately 800 μm in width and 300 μm in height). In some embodiments, the sphere culture chambers are coated in a polyethylene glycol blocking agent. In some embodiments, the mixing layer in configured to generate all possible combinations with a plurality of different mixing ratios of chemical chemicals for a plurality of different chemicals. In some embodiments, the device is configured to generate at least 2 (e.g., at least 3, 4, 5, 6, 7, 8, 9, 10 or more) different mixing ratios of the chemicals. In some embodiments, the biased tree mixers comprise channels with different widths. In some embodiments, channels on both sides are designed to be wider than those at the center. In some embodiments, mixers comprise channels at the last stage that are of the same dimension. In some embodiments, the device is configured to load a plurality of cells in a single cell loading step. In some embodiments, the biased tree mixers generate a log-scale concentration gradient of chemical concentrations. In some embodiments, the sphere culture chamber is configured for cell co-culture. In some embodiments, the device is constructed of PDMS, PS, PMMA, COC, or a combination thereof. In some embodiments, the device comprises spheroid culture chambers of a plurality of different sizes. In some embodiments, the spheroid culture chambers are configured for adherent or suspension culture. In some embodiments, the spheroid culture chambers comprise a center sphere culture chamber, a ring chamber surrounding the center sphere culture chamber, and a thin gap connecting the culture chamber and the ring chamber. In some embodiments, the thin gap is approximately 5 μm in height and 50 μm in length. In some embodiments, a plurality of micro-pillars (e.g., at least 20) are deployed in the thin gap. In some embodiments, the micro-pillars are approximately 5 μm in height and 25 μm in side length. In some embodiments, the chemical inlets route chemicals to a plurality of vias in different rows. In some embodiments, the vias and drug inlets are arranged to allow for mixing of all combinations of different chemicals (e.g., N×N array for N different chemicals).

Further embodiments provide a system, comprising: a) device described herein; and b) an analysis component configured to analyze cell properties of cells cultured in the device. In some embodiments, the analysis component comprise a computer process and/or computer software configured to input images of cells cultured in the device, calculate the effect of a plurality of the chemicals on the cells, and generate a synergistic index of each of the cells in each of the sphere culture chambers. In some embodiments, the synergistic index is a measure of synergist activity between a combination of two or more chemicals. In some embodiments, the property is cell viability. In some embodiments, the analysis component comprises an imaging device (e.g., camera).

In yet other embodiments, the present disclosure provides a method, comprising: a) contacting a plurality of cells with the system described herein; b) culturing the cells in the presence of a plurality of different concentrations of an least two chemicals; and c) calculating a synergistic index for the chemicals. In some embodiments, the cells are cancer cells. In some embodiments, the chemicals are chemotherapy drugs or test compounds. In some embodiments, the method is a high throughput method.

Additional embodiments will be apparent to persons skilled in the relevant art based on the teachings contained herein.

### DESCRIPTION OF THE DRAWINGS

FIG. 1 shows an exemplary equivalent electronic circuit schematic for “Christmas tree” concentration gradient generator system.

FIG. 2 shows COMSOL simulation result of “biased tree” mixer structure. (a) Diagram of chemical mixing simulation result. (b) Quantitative result of the concentration gradient, ranging in 1:1E6, 1:100, 1:10, 1:1, 10:1, 100:1, and 1E6:1.

FIG. 3 shows exemplary drug inlets layout matrix for 16 drugs.

FIG. 4 shows spheroid formation in culture chambers. (a) Laser confocal microscope image of a unit chamber. (b) Size distribution of spheroids formed in the two different-size culture chambers. (c) Microscopy images of sphere forming in sphere culture chambers. (Scale bar =200 μm

FIG. 5 shows exemplary schematics of multiplexed drug combination screening chip. (a) 3 PDMS layers. (b) Top view of the 3 layer design.

FIG. 6 shows schematics of wire routing of 8×4 pins using PADS software, in which blue circles stand for drug reservoirs, and lines stand for microfluidic channels in the routing layer.

FIG. 7 shows an exemplary SUM159 drug combination susceptibility test. (a) Using 20 μM docetaxel, 2 uM doxorubicin, culture media, and 200 uM cisplatin. (b) Example images of 2 spheroids with death rate of 15.8% and 71.2% measured by custom software, respectively. (Scale bar=300 μm

FIG. 8 shows drug combination screening results of culture media and 7 drugs using pancreatic cancer patient-derived cell lines.

FIG. 9 shows drug mixing tests using PBS, Fluorescein (green), tetramethylrhodamine (red), and DAPI (blue), from left to right. (a) Fluorescent dye mixing image on-chip by overlapping images of brightfield, FITC, TRITC and DAPI. (b) Comparison of fluorescent intensity between experiment (solid lines) and simulation (dashed lines), verifying uniform gradient generation. (Scale bar=500 μm)

FIG. 10 shows heterogeneous drug response between SUM159 and MCF7 breast cancer cell lines.

### DEFINITIONS

To facilitate an understanding of the present invention, a number of terms and phrases are defined below:

As used herein, the term “adherent culture chamber” refers to a well or chamber configured for cells to adhere to. In some embodiments, adherent culture chambers are adherent due to the surface material or coating.

As used herein, the term “suspension culture chamber” refers to a chamber or well that cells are unable or substantially unable to adhere to. In some embodiments, the surface of the suspension culture chamber is coated with a material that prevents or repels cells (e.g., polyHEMA).

The term “sample” is used in its broadest sense. On the one hand it is meant to include a specimen or culture. On the other hand, it is meant to include both biological and environmental samples. A sample may include a specimen of synthetic origin.

As used herein, the term “cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

### DETAILED DESCRIPTION OF THE INVENTION

Provided herein are compositions, systems, and methods for high throughput screening. In particular, provided herein are microfluidic devices for high throughput analysis of multiplex chemical (e.g., drug interactions) across a wide range of concentrations.

Accordingly, provided herein is a scalable, easy-to-handle, high-throughput drug combination screening platform, incorporated with custom software for drug efficacy readout and data analysis. The presented microfluidic design scheme enables screening of all possible pairwise drug combinations from N different drugs. Experiments described below implemented an 8-drug combination platform. Combining 28 drug combinations, 7 mixing ratios, 2 sphere sizes, and 5 replicates, a total of 1,960 drug efficacy screening experiments can be accomplished in a single chip. Using pancreatic cancer patient-derived cell lines, effective drug combination screening for both precision medicine and industrial drug combination development applications was demonstrated.

Conventional in-vitro cancer drug screening are mostly performed on two-dimensional (2D) well-plates as a simple, fast, and cost-effective tool to avoid large-scale animal experiments. However, accumulating evidence on in-vitro cancer studies show that a large number of cellular features and gene expression are impaired in a 2D culture environment, which makes it less reliable to make accurate clinical decisions. Three-dimensional (3D) cell culture systems have emerged as potentially better models in mimicking the in-vivo tumor microenvironment, and have become increasingly popular in drug screening studies.

Accordingly, in some embodiments, devices of embodiments of the present disclosure provides microfluidic devices for high throughput drug screening that allows for analysis of a plurality of chemicals (e.g., drugs or test compounds) at a wide range of concentrations and mixing ratios.

The devices and systems described herein allow for high-throughput drug combination screening of multiple drugs. In contrast, previous microfluidic drug combination screening systems only enabled 2 drugs. The highly multiplexed design enables easy operation. Thousands of cell culture spheres can be formed in single cell loading step. In addition, only a single-pipette step is required for multiple drug combination mixing, which is a time-consuming process for existing robotic arm dispensing system. The “biased tree” mixer structure design described below achieves log-scale concentration gradient generation with a very wide range. Previous microfluidic gradient generation was either limited to linear gradient or required a complex handling procedure. In addition, the use of inlet array layout and routing layer design reduces the number of inlets significantly, which makes it possible for large-scale system integration. Furthermore, the thin gap cell capture scheme and curved substrate profile enables reliable spheroid formation of controllable size. Previous systems were unable to achieve high-throughput spheroid formation with the controllable size.

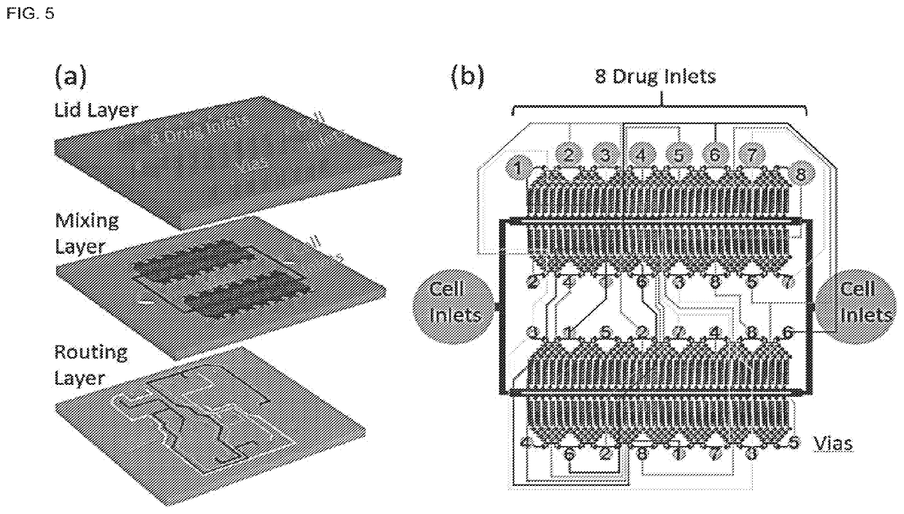

Exemplary devices are shown in FIG. 5. In some embodiments, devices comprise a plurality of layers. In some embodiments, the first or bottom layer is a routing layer that comprises a plurality of microfluidic channels and a plurality of chemical (e.g., drug) reservoirs, wherein the microfluidic channels are in fluid communication with the chemical reservoirs. This layer serves to connect multiple chemical reservoirs for the same chemical (e.g., drugs) or combination of chemicals. In some embodiments, the layout of the routing channels is generated automatically using circuit board design.

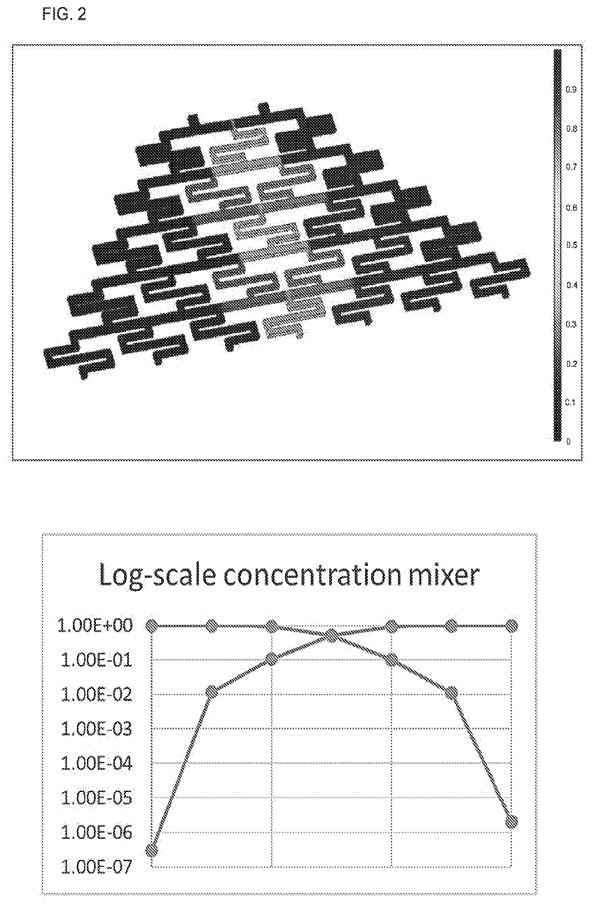

In some embodiments, devices comprise a middle mixing layer. In some embodiments, the mixing layer comprises a plurality of biased tree mixers patterned therein and a plurality of sphere culture chambers. In some embodiments, the biased tree mixers generate different chemical combinations. The biased tree mixer design is shown in FIG. 2.

In some embodiments, the biased tree mixer structure utilizes non-uniform channel sizes to generate a log-scale mixing gradient between two different chemicals. This is achieved by differences in hydraulic resistance of channels with different widths. In some embodiments, channels on both sides are designed to be wider than those at the center. This allows for the generation of wide concentration ratios (e.g., at least 1:1×10⁶) between different chemicals. In some embodiments, mixers comprise channels at the last stage that are of the same dimension. The devices described herein are not limited to the generation of concentration gradients between two different chemicals. In some embodiments, the biased tree mixer design allows for the generation of log-scale concentration gradients for at least two (e.g., at least 3, 4, 5, 6, 7, 8, 9, 10, 12, 14, 16, 20, or more) different chemicals. In some embodiments, devices comprise a plurality of adjacent mixers to allow for greater than two different chemicals. In some embodiments, adjacent mixers share common drug inlets.

In some embodiments, drug mixers are in fluid communication with cell culture (e.g., 2-D or 3-D spheroid) culture chambers. In some embodiments, the sphere culture chambers are coated in a polyethylene glycol blocking agent (e.g., for adherent cell culture). In some embodiments, the device is configured to load a plurality of cells into all the cell culture chambers in a single cell loading step. In some embodiments, the device comprises spheroid culture chambers of a plurality of different sizes. In some embodiments, the spheroid culture chambers are configured for adherent or suspension culture. In some embodiments, the spheroid culture chambers comprise a center sphere culture chamber, a ring chamber surrounding the center sphere culture chamber, and a thin gap connecting the culture chamber and the ring chamber. In some embodiments, the thin gap is approximately 5 μm in height and 50 μm in length. In some embodiments, a plurality of micro-pillars (e.g., at least 20) are deployed in the thin gap. In some embodiments, the micro-pillars are approximately 5 μm in height and 25 μm in side length. In some embodiments, the sphere culture chamber is configured for cell co-culture of a plurality of different cell types (e.g., a cancer cell and a stromal cell). In some embodiments, two different cell types are culture together. In some embodiments, a first cell type is loaded into the ring chamber and a second cell type is loaded into the central chamber so the cells are in fluid communication.

In some embodiments, the device further comprises a lid layer comprising a plurality of chemical inlet holes, a plurality of cell inlet holes, and a plurality of vias. In some embodiments, the vias are microfluidic channels or plastic tubing that connect the layers of the device.

In some embodiments, the microfluidic channels are 100 to 1000 μm in width and 100 to 1000 μm in height (e.g., approximately 800 μm in width and 300 μm in height. In some embodiments, the device is constructed of PDMS, PS, PMMA, COC, or a combination thereof.

The present disclosure is not limited to particular methods for fabricating microfluidic devices. In some embodiments, devices are made from poly-dimethylsiloxane (PDMS).

In some embodiments, layers are made by supplying a negative “master” and casting a castable material over the master. Castable materials include, but are not limited to, polymers, including epoxy resins, curable polyurethane elastomers, polymer solutions (e.g., solutions of acrylate polymers in methylene chloride or other solvents), curable polyorganosiloxanes, and polyorganosiloxanes which predominately bear methyl groups (e.g., polydimethylsiloxanes (“PDMS”)). Curable PDMS polymers are well known and available from many sources. Both addition curable and condensation-curable systems are available, as also are peroxide-cured systems. All of these PDMS polymers have a small proportion of reactive groups which react to form crosslinks and/or cause chain extension during cure. Both one part (RTV-1) and two part (RTV-2) systems are available. Additional curable systems are preferred when biological particle viability is needed.

In some embodiments, transparent devices are desirable. Such devices may be made of glass or transparent polymers. PDMS polymers are well suited for transparent devices. A benefit of employing a polymer which is slightly elastomeric is the case of removal from the mold and the potential for providing undercut channels, which is generally not possible with hard, rigid materials. Methods of fabrication of microfluidic devices by casting of silicone polymers are well known. See, e.g. D. C. Duffy et al., “Rapid Prototyping of Microfluidic Systems in Poly(dimethylsiloxane),” Analytical Chemistry 70, 4974-4984 (1998). See also, J. R. Anderson et al., Analytical Chemistry 72, 3158-64 (2000); and M. A. Unger et al., Science 288, 113-16 (2000), each of which is herein incorporated by reference in its entirety.

In some embodiments, fluids are supplied to the device by any suitable method. Fluids may, for example, be supplied from syringes, from microtubing attached to or bonded to the inlet channels, etc.

Fluid flow may be established by any suitable method. For example, external micropumps suitable for pumping small quantities of liquids are available. Micropumps may also be provided in the device itself, driven by thermal gradients, magnetic and/or electric fields, applied pressure, etc. Integration of passively-driven pumping systems and microfluidic channels has been proposed by B. H. Weigl et al., Proceedings of MicroTAS 2000, Enshede, Netherlands, pp. 299-302 (2000).

In other embodiments, fluid flow is established by a gravity flow pump, by capillary action, or by combinations of these methods. A simple gravity flow pump comprises a fluid reservoir either external or internal to the device, which contains fluid at a higher level (with respect to gravity) than the respective device outlet. Such gravity pumps have the deficiency that the hydrostatic head, and hence the flow rate, varies as the height of liquid in the reservoir drops. For many devices, a relatively constant and non-pulsing flow is desired.

To obtain constant flow, a gravity-driven pump as disclosed in published PCT application No. WO 03/008102 A1 (Jan. 18, 2002), herein incorporated by reference, may be used. In such devices, a horizontal reservoir is used in which the fluid moves horizontally, being prevented from collapsing vertically in the reservoir by surface tension and capillary forces between the liquid and reservoir walls. Since the height of liquid remains constant, there is no variation in the hydrostatic head.

Flow may also be induced by capillary action. In such a case, fluid in the respective outlet channel or reservoir will exhibit greater capillary forces with respect to its channel or reservoir walls as compared to the capillary forces in the associated device. This difference in capillary force may be brought about by several methods. For example, the walls of the outlet and inlet channels or reservoirs may have differing hydrophobicity or hydrophilicity. Alternatively, the cross-sectional area of the outlet channel or reservoir is made smaller, thus exhibiting greater capillary force.

In some embodiments, the present disclosure provides systems comprising the devices described herein and an analysis component configured to analyze cell properties of cells cultured in the device. In some embodiments, the analysis component comprise a computer process and/or computer software configured to input images of cells cultured in the device, calculate the effect of a plurality of the chemicals on the cells, and generate a synergistic index of each of the cells in each of the sphere culture chambers. In some embodiments, the synergistic index is a measure of synergist activity between a combination of two or more chemicals. In some embodiments, the property is cell viability. In some embodiments, the analysis component comprises an imaging device (e.g., camera). In some embodiments, the analysis component detects any desired datable signal (e.g., fluorescent, optical, luminescent, etc.). In some embodiments, the analysis component further comprises a display screen for displaying images and/or results of the analysis.

Embodiments of the present disclosure provide methods of high throughput screening of multiple chemicals (e.g., drugs or test compounds) on a property of a cell (e.g., viability or rate of cell growth). The present disclosure is not limited to a particular cell type. The systems and methods described herein find use with a variety of cell types, including prokaryotic and eukaryotic cells and single cell organisms. In some embodiment, human or mammal cells are utilized (e.g., primary cells, stem cells (e.g., cancer stem cells), immortalized cells, cancer cell lines, etc.).

In some embodiments, molecular properties of cells are analyzed and compared (e.g., in the device via staining for live cells or after removal from the device). In some embodiments, live cells are analyzed. In some embodiments, intact fixed cells are analyzed. In some embodiments, cells are lysed and molecular analysis is performed.

The present disclosure is not limited to particular types of analyses. Examples include, but are not limited to, screening cells for gene expression at the mRNA or protein level (e.g., via reporter genes in live cells or molecular analysis); screening compounds (e.g., drugs) for their effect on cell growth, cell death, viral infectivity, or gene expression; screening viruses for infectivity (e.g., plaque formation); epigenome analysis (e.g., methylation status of genes and/or promoters), protein analysis (e.g., immunoassays such as e.g., single cell Western blot and mass spectrometry analysis), copy-number variations (CNVs) assays, and screening for mutations or polymorphisms (e.g., SNPs).

The present disclosure is not limited to particular analysis methods. Examples include, but are not limited to, sequencing analysis, hybridization analysis, and amplification analysis. Exemplary analysis methods are described herein.

A variety of nucleic acid sequencing methods are contemplated for use in the methods of the present disclosure including, for example, chain terminator (Sanger) sequencing, dye terminator sequencing, and high-throughput sequencing methods. Many of these sequencing methods are well known in the art. See, e.g., Sanger et al., Proc. Natl. Acad. Sci. USA 74:5463-5467 (1997); Maxam et al., Proc. Natl. Acad. Sci. USA 74:560-564 (1977); Drmanac, et al., Nat. Biotechnol. 16:54-58 (1998); Kato, Int. J. Clin. Exp. Med. 2:193-202 (2009); Ronaghi et al., Anal. Biochem. 242:84-89 (1996); Margulies et al., Nature 437:376-380 (2005); Ruparel et al., Proc. Natl. Acad. Sci. USA 102:5932-5937 (2005), and Harris et al., Science 320:106-109 (2008); Levene et al., Science 299:682-686 (2003); Korlach et al., Proc. Natl. Acad. Sci. USA 105:1176-1181 (2008); Branton et al., Nat. Biotechnol. 26(10):1146-53 (2008); Eid et al., Science 323:133-138 (2009); each of which is herein incorporated by reference in its entirety.

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., Life Technologies/Ion Torrent, and Pacific Biosciences, respectively.

Other emerging single molecule sequencing methods include real-time sequencing by synthesis using a VisiGen platform (Voelkerding et al., Clinical Chem., 55: 641-58, 2009; U.S. Pat. No. 7,329,492; U.S. patent application Ser. No. 11/671956; U.S. patent application Ser. No. 11/781166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

Illustrative non-limiting examples of nucleic acid hybridization techniques include, but are not limited to, in situ hybridization (ISH), microarray, and Southern or Northern blot. In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

Different kinds of biological assays are called microarrays including, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes or transcripts (e.g., those described in table 1) by comparing gene expression in disease and normal cells. Microarrays can be fabricated using a variety of technologies, including but not limiting: printing with fine-pointed pins onto glass slides; photolithography using pre-made masks; photolithography using dynamic micromirror devices; ink-jet printing; or, electrochemistry on microelectrode arrays.

Southern and Northern blotting is used to detect specific DNA or RNA sequences, respectively. DNA or RNA extracted from a sample is fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

Nucleic acids may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

The methylation levels of non-amplified or amplified nucleic acids can be detected by any conventional means. For example, in some embodiments, Methylplex-Next Generation Sequencing (M-NGS) methodology is utilized. In other embodiments, the methods described in U.S. Pat. Nos. 7,611,869, 7,553,627, 7,399,614, and/or 7,794,939, each of which is herein incorporated by reference in its entirety, are utilized. Additional detection methods include, but are not limited to, bisulfate modification followed by any number of detection methods (e.g., probe binding, sequencing, amplification, mass spectrometry, antibody binding, etc.) methylation-sensitive restriction enzymes and physical separation by methylated DNA-binding proteins or antibodies against methylated DNA (See e.g., Levenson, Expert Rev Mol Diagn. 2010 May; 10(4): 481-488; herein incorporated by reference in its entirety).

In some embodiments, gene expression or other protein analysis (e.g., detection of cell surface antigens) is performed using immunoassays or mass spectrometry.

Ilustrative non-limiting examples of immunoassays include, but are not limited to: immunoprecipitation; Western blot; ELISA; immunohistochemistry; immunocytochemistry; flow cytometry; and, immuno-PCR. Polyclonal or monoclonal antibodies detectably labeled using various techniques known to those of ordinary skill in the art (e.g., colorimetric, fluorescent, chemiluminescent or radioactive) are suitable for use in the immunoassays. Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

### EXAMPLES

The following examples are provided in order to demonstrate and further illustrate certain embodiments and aspects of the present disclosure and are not to be construed as limiting the scope thereof.

### Example 1

Microfluidic filter structure for cell capture and sphere formation The chip comprises 1960 3D sphere culture units (28 combinations×7 concentration ratios×2 sphere sizes×5 replicates). Each culture unit is composed of a center sphere culture chamber, a ring chamber surrounding the center sphere culture chamber, and a thin gap (5 um in height, 50 um in length) connecting the two chambers. A total of 20 octagon micro-pillars (5 um in height, 25 μm in side length) were sparsely deployed between the PDMS thin gap to provide mechanical support and prevent collapse (FIG. 4(a)). To facilitate 3D cell culture, Pluronic F-108 was loaded to the device 12 hours before cell loading to create non-adherent surface. Since drug efficacy varies with tumor size (James, Keith, et al., Journal of the National Cancer Institute, 91.6 (1999): 523-528), it is also important to take sphere sizes into consideration. By regulating chamber dimensions, form spheres of two different sizes were formed (large: 238±16 μm, small: 124±11 μm), respectively, for comprehensive drug screening (FIG. 4(b)). In the cell loading process, cells are captured at the 5 μm thin gap in each individual chamber and aggregate to form spheroids at Pluronic F-108 coated rounded substrate in central octagonal chamber after 1 day cell culture (FIG. 4(c)).

### Microfluidic Tree Structure as Log-Scale Concentration Gradient Generator

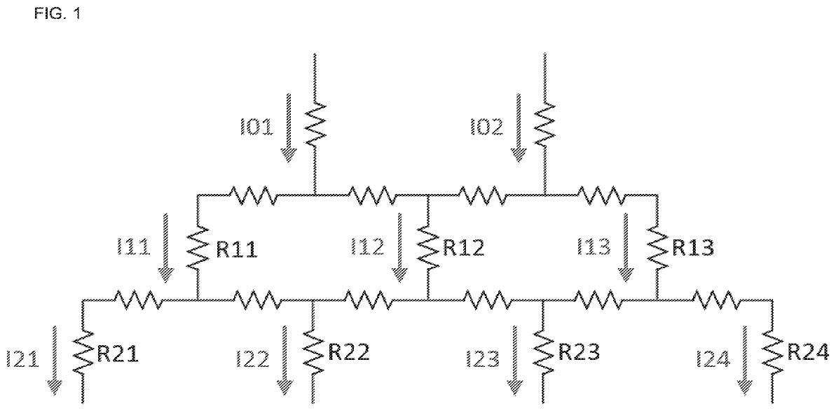

In conventional “Christmas tree” concentration gradient generators, solutions containing different compounds are introduced from the top inlets and flowed through the microchannel network. The fluid streams are combined in each branch channel stage, yielding mixture of distinct compositions, and split to next stage. Finally, a concentration gradient is generated across the last stage of branch channel (Chung, Bong Geun, et al., supra). The splitting ratio of the flow at each stage is determined by the flow resistance, e.g., dimension of the meander channels, which could be understood with an equivalent “electronic circuit” model. Each microfluidic channel in this “Christmas tree” is seen as a resistor in circuit, while the fluid flow is seen as electric current. As is shown in FIG. 1, two different solutions, drug A and drug B, are introduced through I01 and I02. The flow resistance of all horizontal channels is ignored, since they are designed to be two orders of magnitude smaller than that of the vertical channels. According to Ohm's law, one can derive the current flowing through the middle channel stage,

$${I}_{11} = \frac{{R}_{12}}{{R}_{11} + {R}_{12}} \cdot {I}_{01} \\ {I}_{12} = \frac{{R}_{11}}{{R}_{11} + {R}_{12}} \cdot {I}_{01} + \frac{{R}_{13}}{{R}_{12} + {R}_{13}} \cdot {I}_{02} \\ {I}_{13} = \frac{{R}_{12}}{{R}_{12} + {R}_{13}} \cdot {I}_{02} \qquad ( 1 )$$

If one assumes the microfluidic structure is symmetric, and the same pressure is applied to drive drug A and drug B. We have I₁₁=I₁₃, and I₁₂=0.5I₀₁+0.5I₀₂. The concentration flowing through any resistor is given by:

$${C}_{mixer} = \frac{{\sum }_{i = 1}^{n} {I}_{i} \cdot {C}_{i}}{{\sum }_{i = 1}^{n} {I}_{i}}$$

The same analysis is applied for the next channel stage,

$${I}_{21} = \frac{{R}_{22}}{{R}_{21} + {R}_{22}} \cdot {I}_{11} \\ {I}_{22} = \frac{{R}_{21}}{{R}_{21} + {R}_{22}} \cdot {I}_{11} + \frac{{R}_{23}}{{R}_{22} + {R}_{23}} \cdot {I}_{12} \\ {I}_{23} = \frac{{R}_{22}}{{R}_{22} + {R}_{23}} \cdot {I}_{12} + \frac{{R}_{24}}{{R}_{23} + {R}_{24}} \cdot {I}_{13} \\ {I}_{24} = \frac{{R}_{23}}{{R}_{23} + {R}_{24}} \cdot {I}_{13} \qquad ( 2 )$$

If all the microfluidic channels are designed to be of the same size (width, height, length), a linear concentration gradient is established in the last stage. However, in some cases, drug combination therapy is most effective when the concentration ratio of the two drugs is 1:100, or even smaller. In conventional multi-stage mixer systems, the compound concentration only covers one order of magnitude. Larger concentration ranges are required to obtain a comprehensive drug efficacy screening.

The devices described in this example utilize a “biased tree” structure with non-uniform channels sizes to achieve a log-scale mixing ratio gradient between two different compounds. From equation (2), there are 2 components in I₂₂: I₁₁ and I₁₂, among which I₁₂ contains 50% drug A and 50% drug B, while I₁₁ contains 100% drug A. In order to decrease the concentration of drug B in I₂₂, the flow from I₁₁ through R₂₂ is increased, while the flow from I₁₂ through R₂₂ is decreased. According to equation (1), I₁₁ is proportional to

$$\frac{{R}_{12}}{{R}_{11} + {R}_{12}} .$$

One can increase I₁₁ by increasing R₁₂ and decreasing R₁₁. The same method applies to I₂₂, that it requires increasing R₁₂ and decreasing R₁₃. According to the Hagen-Poiseuille equation, hydraulic resistance of a channel is approximately inversely proportional to squared channel width (Mortensen, et al., Physical Review E 71.5 (2005): 057301). Therefore, the channels for R₁₁ and R₁₃ are designed to be wider than that for R₁₂. Similar calculations are also be applied to the following channel stages where more branch channels are incorporated. In general, channels on both sides are designed to be wider than those at the center. Thus the flow of certain compounds is guided to its own side, with only a small portion mixed with the other compound. In this way, various concentration ratio, as large as 1:1E6, are obtained at mixer outlets.

Another important feature of the devices described in this example is that the seven channels at the last stage of the mixer array are of the same dimension. The rationale for this design is that the downstream microfluidic structure also contributes to the equivalent resistance of the last stage of the “biased tree” mixer. Even if the channel dimensions are carefully designed, the equivalent resistance seen from the previous stage has some variation. In return, these channels at the last stage of the mixer also have an influence on the flow resistance of the downstream microfluidic structures. Cells are loaded from a cell inlet to cell culture chambers, which is the downstream hydraulic system for the “biased tree” mixer. Channels of the same dimension help balance the cell loading and form tumor spheres of uniform size.

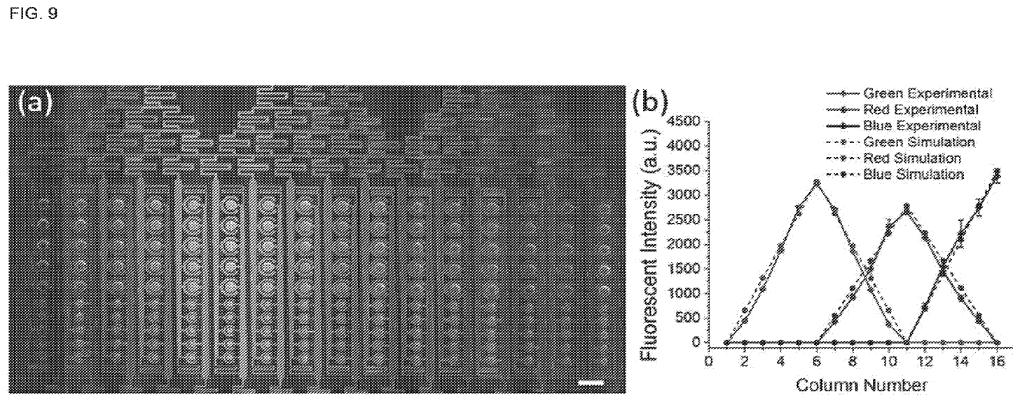

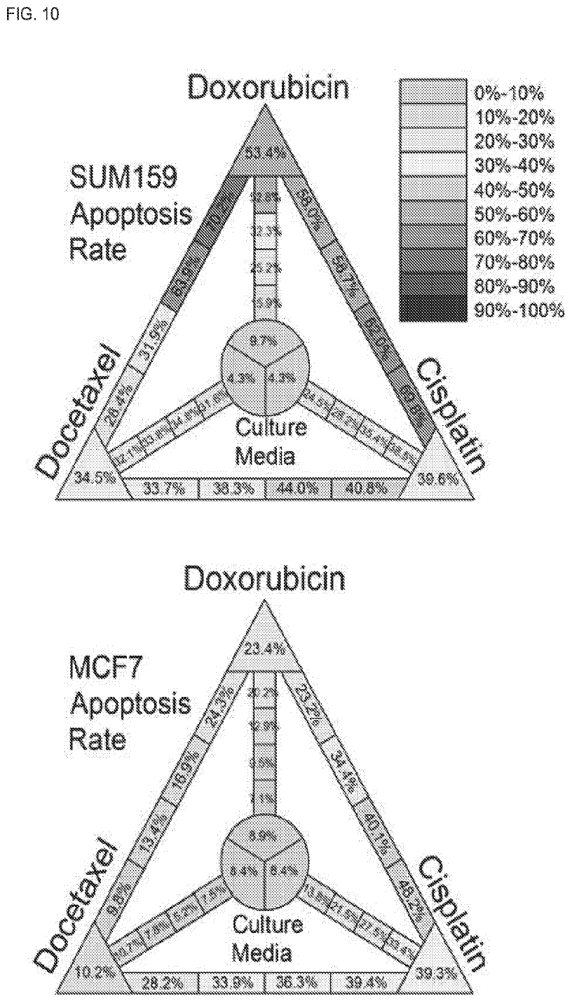

The COMSOL simulation result of the mixer structure is shown in FIG. 2(a), in which the channel dimensions are designed to be symmetric. To achieve the proper range of mixing ratios, the center channels are 60 μm in width, the channels on the edge are 120 μm in width, while the channels in between are 90 um in width. As a result, seven concentration ratios between drug A and drug B are achieved at a log-scale gradient, ranging in 1:1E6, 1:100, 1:10, 1:1, 10:1, 100:1, and 1E6:1 (FIG. 2(b)), which is desirable for drug screening platforms. Uniform mixing was verified using PBS (no color) and three fluorescent dyes (green, red, and blue) (FIG. 9).

### Mixer Array and Drug Inlets Layout Design

In some embodiments, devices comprise “vias” or vertical interconnected ports between layers. In some embodiments, vias are punched holes in the PDMS devices to connect the routing layer, mixing layer and lid layer. In some embodiments, in the routing layer, certain drug solutions loaded to a drug inlet are guided to multiple “vias” in different rows through microfluidic channels. Then, the drug solution flows into mixing layer through these “vias”, and combine with another adjacent drug solution.

By deploying the “biased tree” mixer structures side by side, a log-scale concentration gradient between multiple drugs is generated. If all the 120 combinations of 16 drugs are to be screened, at least 120×2=240 drug inlets are needed for all the biased tree mixers, which sometimes utilizes very complicated microfluidic interface tubing system. In addressing this issue, adjacent mixers are designed to share a common drug inlet, so that the number of inlets is reduced by half. However, this design uses special arrangement of the inlet array to guarantee the adjacent drug pairs covers all the possible combinations. For example, if one has drugs, the drug inlets can be arranged as follows:

In which, number 1-4 stand for 4 different drugs, and there is a “biased tree” mixer between each number pairs to generate concentration gradient. However, the arrangement becomes much more complicated when the drug number becomes larger, for example, the computational complexity could reach 10²⁶ possible permutations when the drug number is 16. As a scalable design, the inlets layout requires a general solution to make it possible for even more drugs included.

This is exemplified with the following:

Use number 1˜N to fill in a N×N table. If one defines the combination of horizontally adjacent numbers as a “pair”, N×(N−1) pairs result in the table. Define “1, 2” and “2, 1” are the different pair. To fill in table, let:
Each row contains 1˜N non-repeating numbers.
All of the N×(N−1) pairs are non-repeating, as well as cover all the possible combinations.
   Since all the requirements are made on adjacency relationship, an “adjacency relationship matrix” is used to fill in the table when the number goes up.

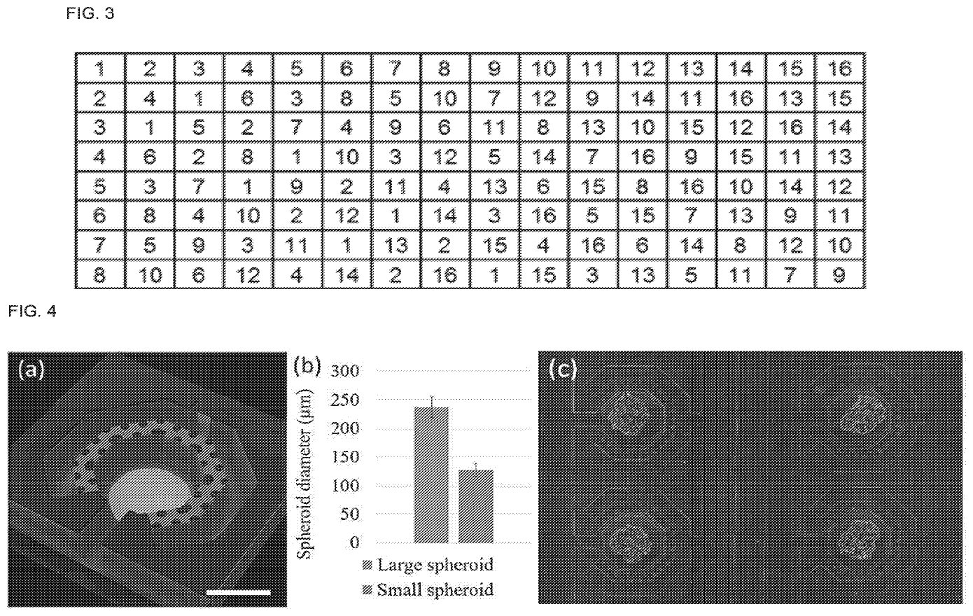

As a demonstration, the matrix for 16 drugs is shown in FIG. 3.

In some embodiments, since all the requirements are made on number adjacency relationship, an “adjacency matrix” is used to help filling in the table when the number goes up. Take the adjacency matrix for N=6 as example:

There are 6×6=36 entries in this adjacency matrix. Each entry represents the existence of certain adjacent number pairs in the original Sudoku table. For example, if one puts an “x” in the entry located at row2 column3, it means that the combination of “2-3” already exist in the original table. Similarly, an entry in row, 5 column 1 means that “5-1” is covered. In this case, if all the entries in this “adjacency matrix” are filled with a “x”, its corresponding table is a good solution. Diagonal entries don't exist and are ignored.

To fill in this table, one first assumes this n*n array should be symmetric. In this way, it is convenient to guarantee “m-n” and “n-m” exist at the same time due to symmetry. Also, it is easy to proof that the first row and first column are arbitrary. In some embodiments, 1-6 are filled in the original Sudoku table as follows:

So its corresponding adjacency matrix becomes:

One keeps filling in the entries in third line along diagonal direction.

Taking advantage of the symmetry of the table, one can derive:

Finally, the 5^{th} line is filled along diagonal direction, obtaining the full solution to 6*6 adjacency table as follows:

The same method using an “adjacency matrix” is applied any number for “N.”.

### Multiple PDMS Layers for Drug Mixing and Routing

As described above, there are N/2 inlets for each drug. It is still labor intensive to load all the N drugs into N² /2 drug reservoirs. In order to minimize the tubing number to N, a three-layer chip design inspired from multilayer circuit board design is used. The three PDMS layers are routing layer, mixing layer, and lid layer from bottom to top (FIG. 1a). The three PDMS layers were fabricated separately using standard soft lithography with silicon wafers as the mold, and then aligned and bonded as FIG. 5(a), (b) after plasma treatment, 6 mm holes are punched through the three PDMS layers as drug reservoirs.

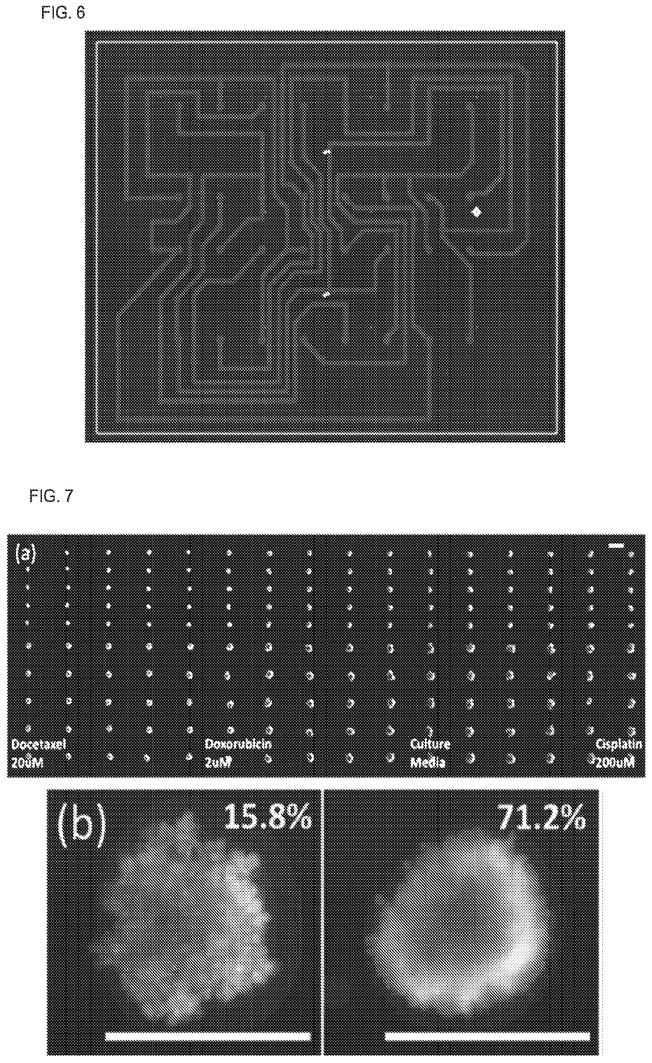

In order to reduce the pipetting steps, a total of N microfluidic channels (800 μm in width, 300 μm in height) are implemented in the routing layer to connect multiple drug reservoirs for the same drug. Through these routing channels, each drug compound is automatically dispensed to all the drug inlets with only single pipetting step. In some embodiments, the layout of the routing channels is generated automatically using circuit board design software (FIG. 6). Since the flow resistance of the routing channels is very small, the drug solution fills all the drug reservoirs in seconds once a drug is loaded to any of the drug reservoirs. For the routing layer, only one mask was used to fabricate the SU8 master mold for the routing channels.

In the mixing layer, different drug compounds are combined in previously mentioned “biased tree” mixers that are patterned on it, together with sphere culture chambers. Three masks were used to fabricate the SU8 master mold for mixing layer: the first mask is for thin gap (5 μm height) with micro-pillars; and the second mask defines patterns for the main microfluidic channels and sphere culture chambers (100 μm height); while the third mask is a dark-field mask with clear patterns of the central sphere culture chamber. The purpose of the third mask is to generate a rounded chamber bottom profile for better cell aggregation and sphere formation. SU8-2010 was spun at 1000rpm for 30 seconds above the first two SU8 layers. Since the average SU8 thickness at this spinning rate is thinner than then second SU8 layer (100 μm), it was not able to cover all the surface. However, SU8-2010 forms a rounded profile above the octagon sphere culture chamber patterns due to surface tension. The curvature is dependent on viscosity, surface properties of the patterned surface, as well as pattern dimensions. Using this method, a curved surface with maximum thickness of 15 μm was generated.

The lid layer PDMS covers the mixing layer to form a closed microfluidic system. The mixing PDMS layer was flipped to face upward before bonding to lid layer, so that cells are captured at the 5 μm thin gap in each individual chamber. After 1 day of culture, cells fell on Pluronic F-108 coated rounded substrate in the central octagonal chamber and aggregate to form spheroids.

### Drug Combination Screening Result

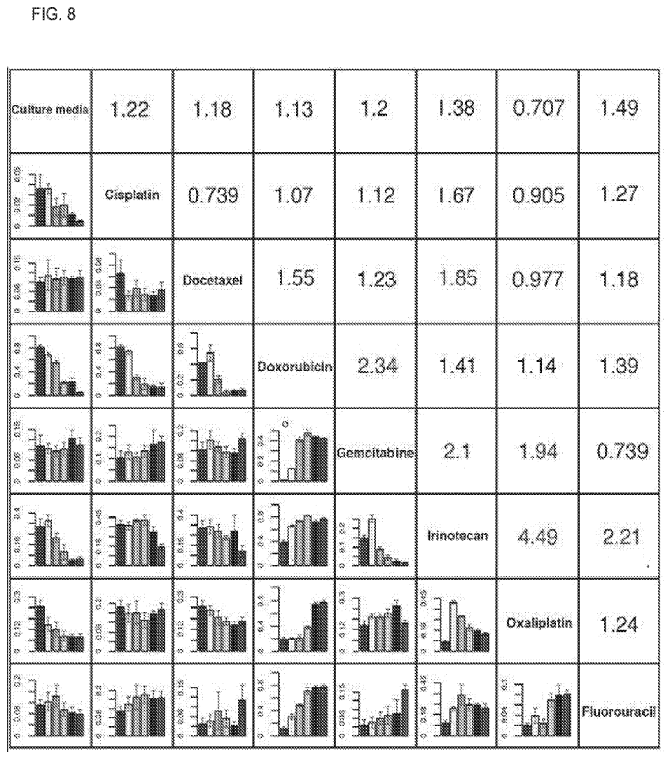

Drug combination screening was performed on pancreatic cancer patient-derived cell lines. The drug-treated cells were stained with live/dead staining assay (calcein AM/ethidium homodimer-1) as cell viability readout, in which live cells are stained with green fluorescence and dead cells with red fluorescence. Drug efficacy was calculated using the fluorescent intensity ratio of live cells to dead cells. A custom program was developed to analysis drug efficacy in a high-throughput manner (FIG. 7). To quantify the coactive effect of various drug combinations, a “synergistic index” is defined by normalizing the cell death rate of the most effective mixing ratio to that of the linear interpolation of single drugs (FIG. 8). The combinations identified with high synergistic indexes (highlighted in red) correlated well with literature (Visk et al., supra; Macarron et al., supra; Du et al., supra; US Food and Drug Administration (2010), supra), among which the highly synergistic irinotecan+oxaliplatin and gemcitabine+oxaliplatin combinations are FDA approved drugs. The non-synergistic combinations (highlighted in blue) such as cisplatin+oxaliplatin and gemcitabine+fluorouracil may result from their similar mechanism of action.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1. A microfluidic device, comprising:
i) a routing layer comprising a plurality of microfluidic channels and a plurality of chemical reservoirs, wherein said microfluidic channel are in fluid communication with said chemical reservoirs;
ii) a mixing layer comprising a plurality of biased tree mixers comprising microchannels of a plurality of different widths patterned therein and a plurality of sphere culture chambers; and
iii) a lid layer comprising a plurality of chemical inlet holes, a plurality of cell inlet holes, and a plurality of vias.

2. The device of claim 1, wherein said vias are microfluidic channels, punched holes, or tubing that connect said lid, said mixing layer, and said routing layer.

3. The device of claim 1, wherein said routing layer forms the bottom layer of said device, said mixing layer forms the middle layer of said device, and said lid layer forms the top layer of said device.

4. The device of claim 1, wherein said microfluidic channels are 100 to 1000 μm in width and 100 to 1000 μm in height.

5. The device of claim 4, wherein said microfluidic channels are approximately 800 μm in width and 300 μm in height.

6. The device of claim 1, wherein sphere culture chambers are coated in a polyethylene glycol blocking agent.

7. The device of claim 1, wherein said mixing layer in configured to generate all possible drug combinations with a plurality of different mixing ratios of said chemicals for a plurality of different chemicals.

8. The device of claim 7, wherein said device is configured to generate at least 3 different mixing ratios of said chemicals.

9. The device of claim 7, wherein said device is configured to generate at least 5 different mixing ratios of said chemicals.

10. The device of claim 1, wherein said device is configured to load a plurality of cells in a single cell loading step.

11. The device of claim 1, wherein biased tree mixers generate a log-scale concentration gradient of chemical concentrations.

12. The device of claim 1, wherein said spheroid culture chambers comprise a center sphere culture chamber, a ring chamber surrounding said center sphere culture chamber, and a thin gap connecting said culture chamber and said ring chamber.

13. The device of claim 12, wherein said thin gap is approximately Sum in height and 50 um in length.

14. The device of claim 12, wherein a plurality of micro-pillars are deployed in said thin gap.

15-18. (canceled)

19. The device of claim 1, wherein said device comprises spheroid culture chambers of a plurality of different sizes.

20. The device of claim 19, wherein each of said chambers comprises at least 20 micro-pillars.

21. The device of claim 19, wherein said micro-pillars are approximately 5 um in height and 25 μm in side length.

22. The device of claim 1, wherein said chemical inlets route chemicals to a plurality of vias in different rows.

23. (canceled)

24. A system, comprising:
a) the device of claim 1; and
b) an analysis component configured to analyze cell properties of cells cultured in said device.

25-28. (canceled)

29. A method, comprising:
a) contacting a plurality of cells with the system of claim 24;
b) culturing said cells in the presence of a plurality of different concentrations of at least two chemicals; and
c) calculating a synergistic index for said at least two chemicals.

30-32. (canceled)
